(19) United States
(12) Patent Application Publication
(10) Pub. No.: US 2002/0151760 A1
(43) Pub. Date: Oct. 17, 2002

(54) Magnetic therapy devices and methods

(76) Inventor: Sumathi Paturu, Pleseant Grove, AL (US)

Correspondence Address: DR. SUMATHI PATURU, M.D., 207 BROOKE-LYN TERRACE, PLEASANT GROVE, AL, 35127, US

(21) Appl. No.: 09/866,330

(22) Filed: May 24, 2001

Related U.S. Application Data
(63) Continuation-in-part of application No. 09/834,436, filed on Apr. 12, 2001.

(51) Int. Cl. A61N 1/00

(52) U.S. Cl. 600/15; 600/13; 600/14

(57) ABSTRACT

Static and electromagnetic therapeutic devices are disclosed for increasing the blood circulation to areas of the body subject to magnetic field induction. The therapeutic devices are useful in the treatment of various diseases and aliments of the human body, such as erectile dysfunction, peripheral vascular disease, cerebral insufficiency and certain vascular pathologies. The disclosure contemplates that the beneficial effects of the induced magnetic fields are the result of the interaction of certain constituents of the blood with the magnetic fields.

### BACKGROUND

This application is a continuation-in-part of U.S. patent application filed on Apr. 12, 2001, entitled Magnetic Therapy Devices and Methods, by Dr. Sumathi Paturu, to which an U.S. application Ser. No. 09/834,436 was assigned. This invention relates generally to the use of magnets in the treatment of human diseases and ailments and devices for carrying out this treatment.

The study of magnetic therapy to treat human disease can be traced back as far as the early 16th century. Over the years, magnetic therapy has been alleged as a cure for diverse diseases and ailments ranging from cancer to chronic pain. The popularity of magnetic therapy continues today. However, despite the prevalence and popularity of magnetic therapy treatments, the physiological effects of magnetic therapy is still unsettled.

Magnetic fields have been historically described in relation to electric current. This relationship to electric current forms the basis of understanding the properties of magnets. All atoms are composed of protons and neutrons, which reside in the nucleus of the atom, and electrons which move rapidly about the nucleus of the atom. As the electrons are negatively charged, each electron generates its own magnetic moment, or magnetic dipole. These magnetic dipoles can be oriented in either of two opposing directions. However, not all atoms demonstrate magnetic properties. This is because many atoms have electrons that are paired with electrons of opposite magnetic dipoles, the net effect being the cancellation of the magnetic dipoles. These atoms are referred to as diamagnetic. Other atoms have unpaired electrons and possess a net magnetic dipole. These atoms do exhibit magnetic properties and are referred to as paramagnetic. Iron is an example of a paramagnetic atom. However, in some cases, the individual magnetic dipoles behave cooperatively and align themselves in the same direction to form magnetic domains. The compounds composed of these atoms demonstrate strong magnetic properties and are referred to as ferromagnetic. Ferromagnetic compounds include iron, cobalt, nickel, samarium, dysprosium and gadolinium.

Magnets always exist as dipoles, with a north pole and a south pole. Magnetic filed lines emerge from the north pole and converge at the south pole. The force of a magnetic field line is known as the magnetic flux and is measured in weber (w). The strength of a magnetic field, or magnetic flux density, is the number of magnetic field lines passing through a unit area and is measured in Telsa (T), or gauss (g).

There are two types of magnetic therapy: electromagnetic therapy and static magnetic field therapy. The types of magnetic fields generated in each of these types of therapy can be different. For example, electromagnetic therapy can employ a pulsating magnet field which allows the strength of the magnetic field to be regulated by controlling the flow of current, while in a static magnetic field the strength of the magnetic field does not vary. Electromagnetic therapy is based on the principle discovered by Michael Faraday that described the relationship between the movement of a magnetic and an electric field (electromagnetic induction). Faraday observed that passing a magnet in and out of a conducting electric coil produced voltage.

It has been known for some time that electrical activity in some form is involved in many aspects of human physiology. For instance, electrical activity has been measured during the regeneration of bone. In addition, it is well documented that many cellular responses are dictated by electrical gradients generated in the cell (for example, nerve cells). Therefore, it is possible that exposure of the human body to an pulsating electromagnetic field could produce a beneficial physiological response in the body. In fact, several studies have shown beneficial effects of pulsating electromagnetic field therapy in stimulating osteogenesis. The United States Food and Drug Administration has recently approved the use of pulsating electromagnetic field therapy for the treatment of some types of bone fractures

Various mechanism have been proposed for the effects of static magnetic field therapy, but none have achieved widespread acceptance. However, whatever the mechanism, the beneficial effects of static magnetic field therapy could most possibly be the result of increased blood flow to the area of the body treated with the static magnetic field.

### BRIEF DESCRIPTION OF THE DRAWINGS

The following embodiments of the disclosure are explained by the drawings and disclosure that follows.

FIG. 1 is an embodiment of the schemaic drawing of the magnetic device used for research purposes.

FIG. 2 is a full frontal view of an embodiment of an undergarment capable of receiving a magnetic therapy device;

FIG. 3 is a schematic drawing an embodiment of a magnetic therapy device suitable for use with the undergarment illustrated in FIG. 2;

FIG. 4 is a full side view of an embodiment of a magnetic condom.

FIG. 5 is a fall frontal view of an embodiment of a magnetic boot;

FIG. 6 is a full frontal view of the an embodiment of the magnetic boot illustrating the connection to an electrical source;

FIG. 7 is a full frontal view of an embodiment of a magnetic headpiece;

FIG. 8 is a full frontal view of an embodiment of a magnetic headpiece for treating localized regions of the head;

FIG. 9A a schematic drawing of an embodiment of a vascular magnetic cuff for treating various vascular pathology; and

FIG. 9B a schematic drawing of an embodiment of a vascular magnetic cuff used at the time of

FIG. 10A and 10B showing a schematic drawing of an embodiment of the therapeutic magnetic sheath.

### DETAILED DESCRIPTION

Magnetic therapy is a widely practiced and growing alternative treatment for many diseases and ailments in Asia, Europe and North America. Although many double blind clinical studies have documented the beneficial effects of various types of magnetic therapy, there is no satisfactory scientific mechanism for these beneficial effects.

It is well established that magnets can attract various types of metals, including iron. In the body, iron is prevalent in many places, including the blood. Blood cells contain hemoglobin molecules. Hemoglobin molecules function to transport oxygen from the lungs to the tissues of the body. Hemoglobin is composed of four subunits, with each subunit containing one molecule of iron, for a total of four iron molecules per hemoglobin molecule. Iron is paramagnetic. As a result, iron possesses a weak magnetization in the direction of an induced magnetic field. In addition, there are other paramagnetic materials present in the blood, including oxygen, sodium and potassium.

The body of a 70 kg man contains approximately 4 grams of iron, with 65%, or about 2.6 grams, being present in the hemoglobin. Therefore, hemoglobin molecules in the blood may contain enough iron to make the red blood cells of the blood responsive to magnetic fields and move, or be pulled, in the direction of an applied magnetic field.

Without being limited to other possible theories, the disclosure contemplates that the therapeutic benefits of static magnetic therapy and electromagnetic therapy that have been observed are mainly the result of increasing the blood circulation in the areas affected by magnetic induction through the attraction of the iron molecules in the hemoglobin molecules. This increased blood circulation may be the result of the attraction of the hemoglobin in the oxygen bound state or the oxygen free state.

In order to demonstrate the affinity of blood cells to magnetic fields, the disclosure has noted a uniquely suitable clinical issue for preliminary study. The penis is a profoundly vascularized organ. In the penis, blood flows into sponge like sinusoids rather than capillary like tubes. In addition, the penile circulation is isolated from the circulation of the body. Therefore, the circulation in the penis can be readily observed without the use of sophisticated radiological devices, and the effects of magnetic induction on the circulation can be observed.

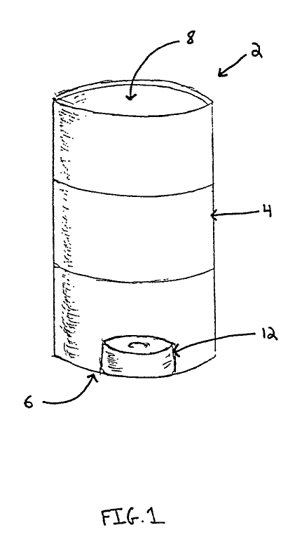

Preliminary evaluation was performed on four human subjects to study the effects of magnetic fields on blood circulation in the penis. Initial studies were conducted using a static magnetic field, but the disclosure should not be read so as to prohibit use of pulsed electromagnetic fields. An embodiment of the device used in the initial studies is shown in FIG. 1. The device 2 has a tubular structure comprising a continues side wall 4 extending the length of the device 2, one closed end 6 and one open end 8 opposite the closed end 6. The length and diameter of the device 2 can be adjusted, depending on the requirements of the user. The side wall 4 in this embodiment is comprised of a magnetic material to provide a magnetic field along the axial length of the penis when the penis is inserted into the device 2. The magnetic material comprising the sidewall 4 may be of any suitable composition, but in this embodiment it is a flexible, rubber magnetic material. A tip magnet 12 is also shown on the interior surface of the closed end 6 with the magnetic field facing the tip of the penis. The tip magnet 12 may be of any composition, but in this embodiment the tip magnet 12 is a button Alnico magnet, which is formed from an alloy of iron, cobalt, aluminum and nickel. A plurality of magnets may be employed. The strength of the magnetic field generated by the magnetic side wall 4 and the tip magnet 12 is such that the magnetic field is effective in increasing blood circulation in the penis and causing an erection (defined as partial or complete engorgement of the penis). The tip magnet 12 may be of higher strength than the magnetic material comprising the side wall 4. In the embodiment shown in FIG. 1 the tip magnet 12 has a strength of 1250 gauss. The device 2 is secured together by any convenient means, such as casting plaster.

Other embodiments of the device 2 are possible. For instance, the device 2 may comprise only the tip magnet 12 with the side wall 4 being comprised of a non-magnetic material. Alternatively, the device 2 may comprise only a side wall 4 of magnetic material. In addition, the side wall 4 may be constructed such that only discrete portions of the side wall 4 contain magnetic material, for use with or without the tip magnet 12. For instance, the magnetic material in the side wall 4 may be placed such that it overlays all or some of the major veins and arteries of the penis.

The penis is inserted into the device 2. The tip of the penis should be placed in close proximity to the tip magnet 12 so that the magnetic field of the tip magnet 12 may exert the maximal pull of the blood towards the tip of the penis, and therefore, through the entire length of the penis. In the initial studies, use of the device for less than 5 minutes was sufficient to achieve penile erection. In several instances, penile erection was observed in time spans less than 1 minute. Additional device can be used to maintain venous occlusion for sustained erection, such as a band like structure encircling the base of the penis.

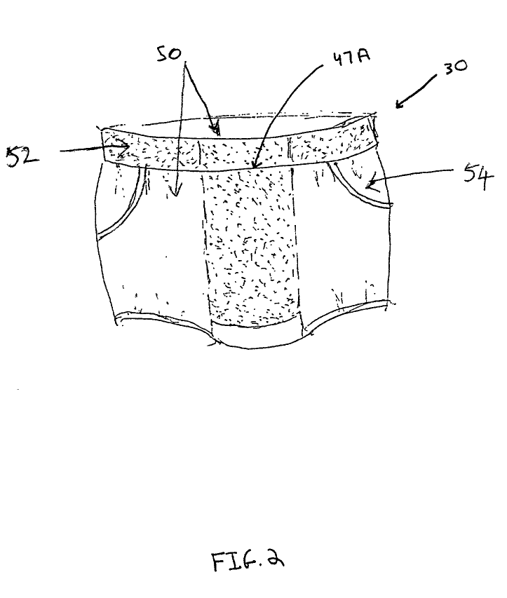

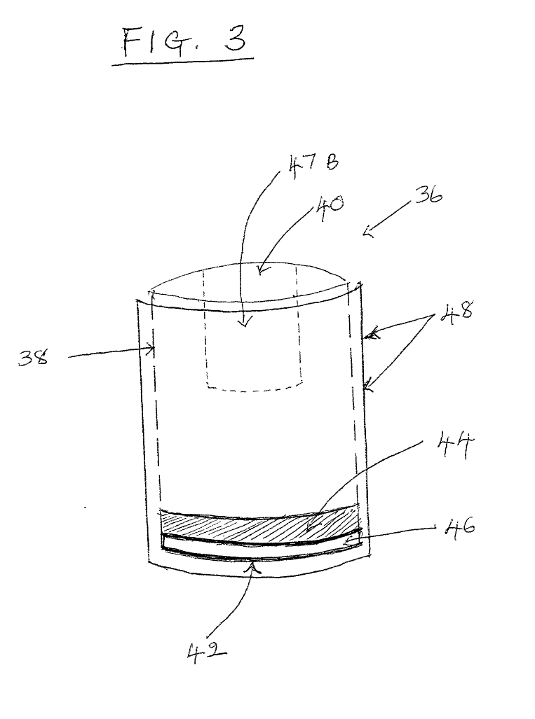

An undergarment 30 incorporating a magnetic element is also disclosed and is shown in FIGS. 2 and 3. The undergarment 30 has an interior and exterior side and may be of either brief or boxer design, with the brief design being illustrated in FIG. 2 In this embodiment, the magnetic element is a detachable pouch 36 of generally tubular shape, comprising a continuous, tubular side wall 38 forming a tubular shape, an open end 40 and a closed end 42 as shown in FIG. 3. In this embodiment, the side wall 38 is formed from flexible magnetic material, such as a thin rubber magnet. A magnet 44 is placed on the interior portion 46 of the closed end 42, with the magnetic field directed toward the open end 40. The magnet 44 may have a steet backing at the closed end 46x in order to increase the strength of the magnet (×32) in the direction of the open end 40 that is towards the tip of the penis.

The pouch 36 can be manufactured in various lengths and diameters depending on the requirements of the user (i.e. depending of the pre-erection size of the penis). Whatever size is selected, it is important that the tip of the penis be in close proximity to the magnet 44 in order for the magnet field produced by the magnet 44 to exert the greatest possible pull on the blood through the length of the penis, which results in engorgement and erection of the penis.

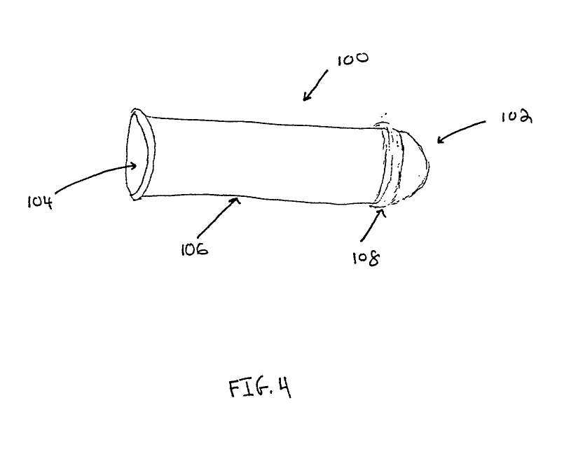

The interior side of the undergarment 30 and the exterior of the pouch 36 have complementary fastening elements 47A and 47B, allowing the pouch 36 to be removably attached to the undergarment 30. In FIGS. 3 and 4, the complementary fastening elements 47A and 47B are velcro patches, but other embodiments are possible, such as buttons, snaps, zippers, etc. The fastening element 47A extends along the length of the center of the front section 50 of the undergarment 30, allowing the user to adjust the height of the pouch 36 as desired. As the pouch 36 is removable from the undergarment 30, different size combinations of the pouch 36 and undergarment 30 can be offered, with the size of the pouch 36 and undergarment 30 being dictated by the requirements of the user. There can be fastening velcro as a waistband inside 52 in the front. It helps moving the pouch 2-3 inches on either side when not needed, thus preventing erection when not desired.

In an alternate embodiment, the pouch 36 may incorporate a sheath of steel foil 48 or similar material on the side of the pouch 36 opposite the complementary fastening element 47B. The function of the sheath 48 is to prevent the magnetic field generated by the magnetic side wall 38 and magnet 44 from producing engorgement and erection of the penis when not desired. When engorgement and erection of the penis is not desired, the penis is removed from the pouch. As such, the penis is effectively shielded from the magnetic fields produced by the side wall 38 and magnet 44.

In an alternate embodiment, the pouch 36 comprises a continuous side wall 38 forming a tubular shape, an open first end 40 and a closed second end 42 as shown in FIG. 3. However, in this embodiment, the side wall 38 is formed from any non-magnetic material instead of the flexible magnetic material described above. The magnet 44 is retained on the interior portion of the closed second end 42, with the magnetic field directed toward the open first end 40. In addition, the pouch 36 may comprise the side wall 38 comprising magnetic material, but omit the magnet 44. In addition, the side wall 38 may be constructed such that only discrete portions of the side wall 38 contain magnetic material, for use with or without the magnet 44. For instance, the magnetic material in the side wall 38 may be placed such that it overlays all or some of the major veins and arteries of the penis.

For the undergarment 30, a pocket 54 is placed for storage of the pouch 36 when not in use. The pocket may have a complementary fastening element to mate with the fastening element 47B on the pouch 36.

As shown in FIG. 4., a magnetic condom 100 is also disclosed. The magnetic condom 100 is of typical construction, comprising a closed tip end 102 and an open end 104, the closed end 102 and the open end 104 being joined by a continuous, tubular side wall 106. The side wall 106 and closed end 102 comprise a thin, durable rubber material in which magnetic particles have been embedded. In this embodiment, the condom 100 also incorporates a ring 108 of magnetic material a few millimeters from the closed end 102 for increased pull of blood to the tip of the penis and throughout the length of the penis. The magnetic material incorporated into the side wall 106 and the ring 108 may be of any effective composition. The strength of the magnetic field created by the side wall 106 and the ring 108 is such that the magnetic field is effective in increasing the blood circulation to the penis. In alternate embodiments, the condom 100 may incorporate magnetic material only in the side wall 106, or only in the ring 108 of condom 100. The condom 100 can be used during intercourse for sustained erection. In sever cases of erectile dysfunction, the condom 100 can be used in conjunction with the device 2 and/or the undergarment 30.

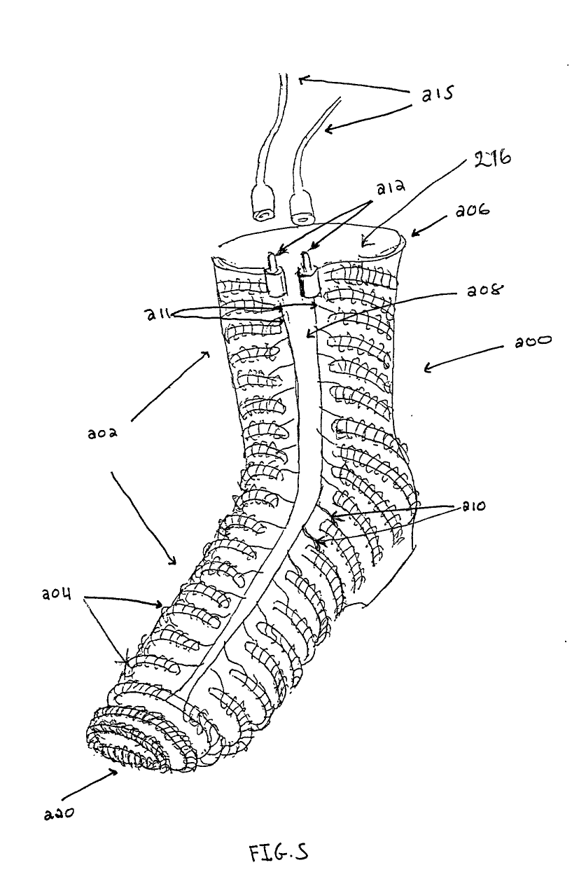

The beneficial effects of magnetic therapy on the blood circulation also has implications that extend to other clinical conditions that are vascular in origin, such as peripheral vascular disease of the legs and feet. Peripheral vascular disease of the lower extremities is commonly found in people suffering from diabetes. A magnetic boot (which for the purpose of specification shall include all types of footwear) for use in the treatment of peripheral vascular disease of the legs and feet is disclosed and shown in FIG. 5. Through the effect of the magnetic field, blood is attracted to the periphery of the legs and feet, thereby increasing peripheral circulation.

The boot 200 is composed of a frame 202. The frame 202 is itself composed of individual frame members 204 secured together and a rim 206. The frame 202 is not completely closed, but is constructed so as to produce an opening 208 in the frame 202. The opening 216 is located on the top of the boot 200 and the opening extends substantially the entire length of the boot 200. The opening 208 allows the user to easily place his foot and/or lower leg into the boot, and provides a comfortable fit and wear to those who suffer from peripheral vascular disease. The frame 202 of the boot 200 can comprise either a magnetic material if static magnetic therapy is desired, or can comprise a malleable iron material if pulsed electromagnetic therapy is desired. The strength of the magnetic field created is such that the magnetic field is effective in increasing peripheral blood circulation.

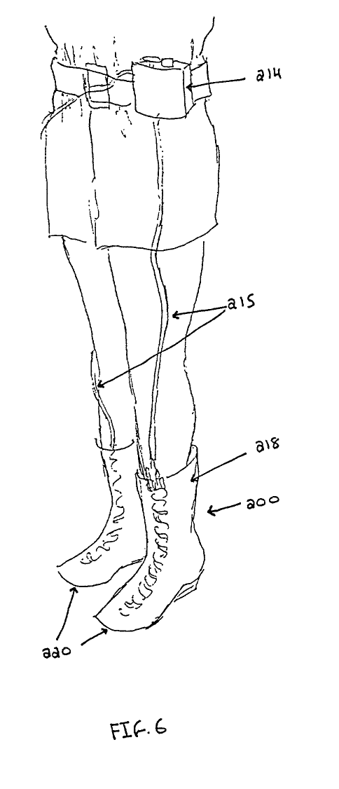

In the case where pulsed electromagnetic therapy is desired, electrical conducting wires 210 are wrapped around the individual frame members 204. The wires 210 extend into the opening 208 where they join main conducting wires 211 which terminate at two electrical connectors 212 secured to the rim 206. The wires 210 and 211 are connected to an adjustable electrical source 214 through electrical cables 215, as illustrated in FIG. 6. The electrical source can activate the electric circuit by allowing electric current to flow through the cables 215 and the wires 210 and 211 Each time an electrical circuit is activated, an electromagnetic field is created around the foot and/or lower leg of the wearer. The frame members 204 function as solenoid due to the effect of the electrical current carried through the wires 210 and increase the magnetic field generated in the wires 210. The electrical source 214 is compact enough so that it can be conveniently carried by the user. No matter whether static or electromagnetic therapy is involved, the boot 200 has a well insulated inner layer (not shown) and outer layer 218, with the frame 202 and wires 210 and 211 being located between the inner layer and the outer layer 218.

The electrical source 214 can be adjusted so that the electrical circuit formed by the wires is activated in discrete temporal patterns. The electrical source 214 may be programmed to deliver electrical pulses which are timed with the user's pulse. For example, a pulse sensor (not shown) may be attached in the groin region of the user such that it can detect the femoral pulse. Each time a femoral pulse is detected, the electrical source 214 is activated, sending electrical current to the wires 210 and 211, thereby inducing an electromagnetic field around the lower legs and/or feet of the user. As discussed above, each time a magnetic field is created, the magnetic field attracts blood cells to the peripheral circulation (which consists largely of capillary beds that have been narrowed due to disease or other factors). When the magnetic fields are activated in coordination with the pulse of the user, the pulling effect of the magnetic field is added to the pushing effect from the pressure of the proximal circulation, resulting in increased blood circulation to the periphery. Also, since the magnetic field attracts the blood cells in coordination with the natural circulation, the blood flow is allowed to enter the venules and into the venous circulation after the plateau of the pulse without the interference of the magnetic field. Therefore, the pulsing of the magnetic field in coordination with the pulse of the user will diminish the possibility of clot formation which may be possible when a continuous magnetic field is applied.

It would also be beneficial if the magnetic field is the strongest at the tip 220 of the boot 200 near the toes and Fore foot as the peripheral circulation in the toes of the fore foot are often the first and most severely affected areas. A stronger magnetic field can be created by increasing the density of the wires 210 around the frame members 204 forming the tip 220 of the boot 200. It may also be desired to increase the strength of the magnetic field along the course of the major arteries, such as the dorsal is pedis artery and the posterior tibial artery. In this case, the density of the wires 210 could be increased along the course of the desired artery.

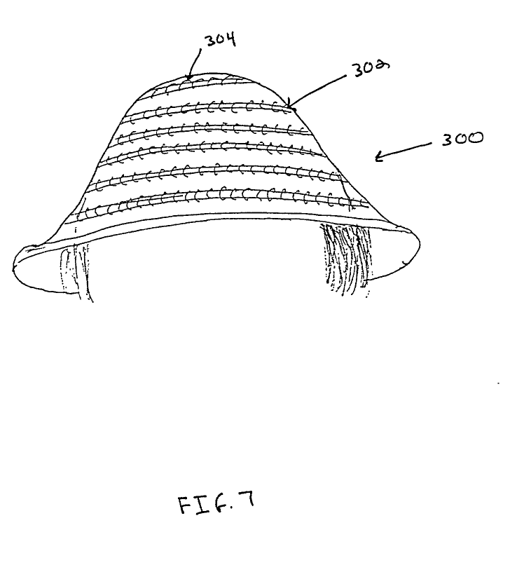

Cerebral vascular insufficiency can also be treated with magnetic therapy. Cerebral vascular insufficiency is often due to atherosclerosis caused by local thrombotic pathology with threatened cerebral infarct or actual cerebral infarct. For generalized cerebral vascular insufficiency, a magnetic headpiece 300 can be used, as illustrated in FIG. 7. The headpiece 300 is of typical construction, comprising an inner side and an outer side, and being adapted for placement on the head of a user to direct magnetic fields through the cranium and into the brain. The magnetic field generated by the headpiece 300 can be either continuous static magnetic field or an electromagnetic field. In either case, strong magnetic fields (on the order of 2000-3000 gauss) will be preferred in order to penetrate the bones of the skull. The magnetic field can be arranged in any useful configuration, such as a spiral or concentric circles.

FIG. 7 illustrates an embodiment of the headpiece 300 incorporating electromagnets. Headpiece conducting members 302 are arranged about and secured to the headpiece 300 in any effective configuration (FIG. 7 shows the headpiece conducting members 302 arranged in close set spiral). Electrical conducting wires 304 are wrapped around the headpiece conducting members 302. If a static magnetic field is desired, the electrical conducting members 302 are formed from a magnetic material, and no conducting wires 304 are used. As noted above, the strength of the magnetic field produced can be varied by varying the density of the wires 304. The wires 304 are connected to an electrical source (not shown) as discussed above in regards to the magnetic boot 200. In addition, the electrical source can be connected to a pulse sensor to control the induction of the magnetic field in coordination with the natural pulse of the user to attain the benefits discussed above.

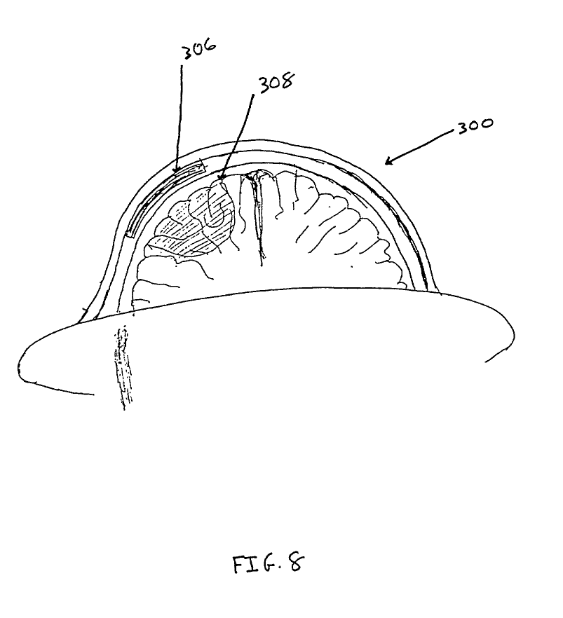

For patients with localized thrombosis and/or threatened or actual infarct of the brain 308, the magnetic field can be localized to prevent Steel syndrome which is a possible complication with generalized magnetic fields. An embodiment of the headpiece 300 illustrating this principle is shown in FIG. 8. The magnetic field can be localized over an effected region in the brain by any convenient means, such as the attachment of a static magnet 306 applied to the inner lining of the headpiece 300. The magnetic 306 can be attached by any means, such as velcro. Alternatively, a magnetic field can be localized over a region of the brain by arranging conducting wires 304 around the conducting members 302 so that a magnetic field is generated only over the area of the brain desired. By localizing the magnetic field to the region of the brain with vascular insufficiency, blood circulation can be increased in these regions by the principles discussed above.

Magnetic therapy may also be applicable to treatment of vascular pathology. Examples of such vascular pathology include those associated with coronary or other atherosclerotic or thrombotic areas with blockage, balloon angioplasty and placement of stents.

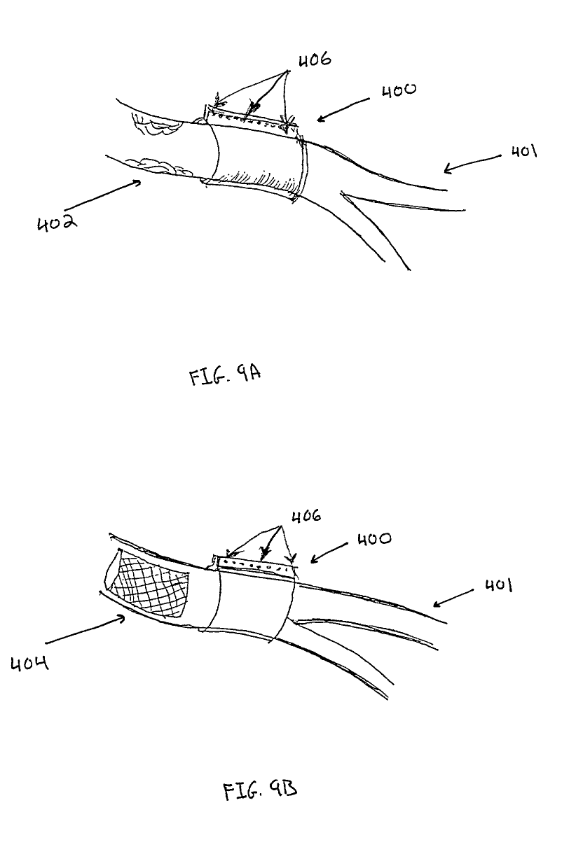

FIGS. 9A and 9B show a vascular magnetic cuff used in the treatment of vascular pathology. In this embodiment, the device is a magnet 400 which is applied around the artery 401 distal to the area~of pathology 402 or distal to the area where a stent 404 is placed. The magnet 400 in the preferred embodiment is a strip of flexible, magnetic material, such as rubber impregnated with magnetic particles, with a first end and a second end and an inner side and an outer side. The magnet 400 is wrapped around the artery 401 at the desired location and secured in place by joining the first end and second end of the magnet 400 together to create seel 406. In this manner the magnet 400 forms a cylindrical structure around the artery. The first and second ends can be joined together by any convenient means, such as cauterization or stapling. The strength of the magnet 400 is such that the magnetic field produced is effective in drawing the circulation distal from the area of pathology 402 or stent 404. The magnet 400 can be of various lengths, depending on the strength of the magnetic field desired, and other factors. The magnet 400 can also be constructed so that there is a gradient of magnetic strength along the magnet 400, with a higher magnetic strength being present in the center area of the magnet 400 and a lower magnetic strength towards the first and second ends of the magnet 400. Therefore, the magnetic field produced by this embodiment of the magnet 400 is greater in the center portion of the magnet 400 than the first and second ends of the magnet 400. The principle is to create a magnetic gradient where the magnetic field gradually increases, reaches a plateau and then gradually decreases. Such a gradient facilitates the unimpeded flow of blood distal to the device.

Due to the placement of the magnet 400 distal to the area of pathology 402 or stent 404, the blood that flows into these areas is attracted to the magnetic field and is pulled distal to the area pathology 402 or stent 404, so that the blood flow does not become stagnant and initiate further thrombosis at the area of pathology 402 or the stent 404. The magnet 400 can be placed around the artery during surgical procedures designed to treat the pathology or insert the Stent 404, or can be applied in the area desired with an instrument similar to an endoscope.

In addition, the magnet 400 may be placed around veins as well as arteries. The principles of operation and the device used are identical to that described for arteries above, except that the magnet 400 is placed proximal to the area desired to be treated.

The description is intended to be illustrative of the construction blocks and system of construction described herein. It should be appreciated that various modifications could be made in the construction blocks, gaskets and system of construction utilizing the construction blocks and gaskets which remain within the scope and teaching of the instant disclosure. The details given herein are to be interpreted as illustrative only and not in a limiting sense.

The principle of electromagnetism can be applied for treating Decubitus ulcers for patients who are bedridden and suffer from ‘difficult to treat or cure’ Decubitus ulcers especially in the sacral area and other places where blood supply is compromised due to their posture (patients with stroke, etc). The involved area can be covered with magnetic sheath with magnetic field strength of 2000-3000 gauss. It can be used over areas with threatened ulcers or as prophylaxis when the bedridden state of the patient is anticipated to be prolonged. Electromagnetic field corresponding to the pulse of the patient is better than static magnetic field. More red blood corpuscles (with more oxygen carrying capacity that is needed to improve ischeamia of the skin and subcuteaneous tissues) are drawn to the periphery when the magnetic field is activated with each pulse. In other words oxygen turn over in the specified area is improved.

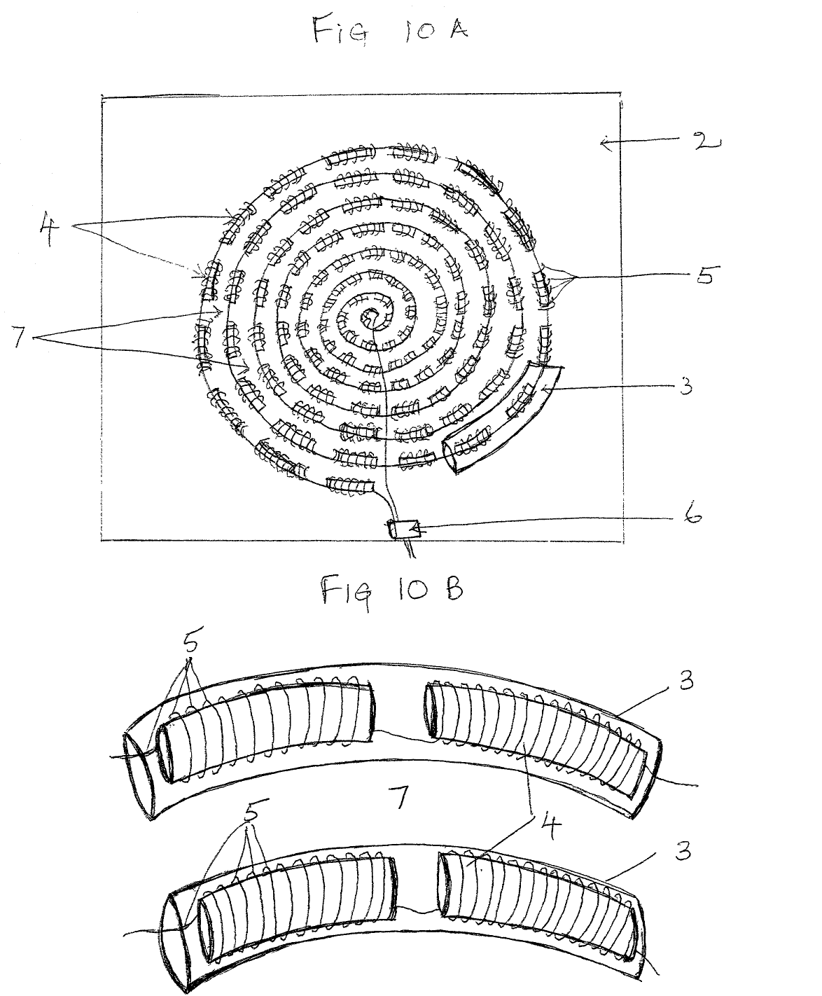

FIG. 10A and 10B illustrate embodiment of the magnetic sheath used for the purpose. It is made up of thin but durable soft fabric 2 for good flexibility. It has a tubular fabric tunnel 3 that starts in the center and spreads out to the periphery in the form of a coil. Malleable iron solenoids 4 which are biconvex but thin in their cross-section are arranged in these tunnels ½ apart. They have conducting wire 5 around them that transverse from one solenoid to the other in the entirety of the coil. The coil is well insulated to prevent thermal or electrical trauma to the patient. Both the ends of the coiling wire are connected to an electrical source (not shown in the picture). The electrical source is also connected to a pulse sensor (can be placed at the level of femoral pulse). The electrical circuit is activated at the onset of the pulse and gets deactivated after the plateau of the pulse (i.e. during the period that corresponds to the diastole of the heart and venous return from the periphery.

To avoid the discomfort of having hard metallic structures under the back of a resting patient, the spaces between the tubular tunnels 7 can be padded with soft material. However, the fabric that covers the surface of the solenoids should be thin to prevent significant impedance of the magnetic field. The coils can be wound more closely to increase the strength of the magnetic field to a range of 2000-3000 gauss. The sheaths are covered on both surfaces with insulating material and over it water proof material. The size of the solenoids can vary depending upon the size of the sheath. The arrangement in the form of a coil and the placement of the solenoids like spokes of a wheel helps the sheath to fold easily to the contour of the back of the patient (sacral area which is most commonly involved). However other arrangements can also be made.

1. A therapeutic device for creating a magnetic field in and around a penis, the device comprising:
a. a closed end, the closed end further comprising an inner and an outer surface, an open end and a side wall, the side wall joining the closed end and the open end together to create a pocket of generally tubular shape defined by the side wall and the closed end; and
b. at least one tip magnet placed on the inner surface of the closed end with the magnetic field directed toward the open end of the device.

2. The device of claim 1 where the strength of the magnetic field produced by the at least one tip magnet is effective in increasing blood circulation in the penis and causing an erection.

3. The device of claim 2 where the at least one tip magnet produces a magnetic field strength greater than 1250 gauss.

4. The device of claim 1 wherein the side wall is comprised of a magnetic material.

5. The device of claim 4 where the strength of the magnetic field produced by the at least one tip magnet and the side wall is effective in increasing blood circulation in the penis and causing an erection.

6. The device of claim 5 where the at least one tip magnet produces a stronger magnetic field than the magnetic field produced by the side wall.

7. The device of claim 6 where the at least one tip magnet produces a magnetic field strength greater than 1250 gauss.

8. The device of claim 1 wherein only discrete portions of the side wall comprise a magnetic material.

9. The device of claim 8 where the discrete portions of the side wall comprising magnetic material corresponds to the course of at least one major artery or vein in the penis.

10. The device of claim 9 where the strength of the magnetic field produced by the at least one tip magnet and the side wall is effective in increasing blood circulation in the penis and causing an erection.

11. The device of claim 10 where the at least one tip magnet produces a stronger magnetic field than the magnetic field produced by the side wall.

12. The device of claim 11 where the at least one tip magnet produces a magnetic field strength greater than 1250 gauss.

13. A condom-like device for creating a magnetic field in and around a penis of, the device comprising:
a. a closed end, an open end and a side wall, the side wall joining the closed end and the open end together to create a pocket of generally tubular shape defined by the side wall and the closed end, the side wall and closed end are comprised of a thin, tear-resistant rubber material containing embedded magnetic particles; and
b. a ring of magnetic material positioned several millimeters from the closed end of the device.

14. The condom-like device of claim 13 where the strength of the magnetic field produced by the side wall, closed end and ring of magnetic material is effective in increasing blood circulation in the penis and causing an erection.

15. The condom-like device of claim 14 where the ring of magnetic material produces a stronger magnetic field than the magnetic material comprising the side wall and closed end.

16. The condom-like device of claim 13 wherein only discrete portions of the side wall and closed end are comprised of thin, tear-resistant rubber material containing embedded magnetic particles.

17. The condom-like device of claim 16 where the discrete portions of the side wall and closed end comprising thin, tear-resistant rubber material containing embedded magnetic particles correspond to the course of at least one major artery or vein in the penis.

18. The condom-like device of claim 17 where the strength of the magnetic field produced by the side wall, closed end and ring of magnetic material is effective in increasing blood circulation in the penis and causing an erection.

19. The condom-like device of claim 13 where the side wall and closed end are comprised of a thin, tear-resistant rubber material not containing embedded magnetic particles.

20. The condom-like device of claim 19 where the strength of the magnetic field produced by the ring of magnetic material is effective in increasing blood circulation in the penis and causing an erection.

21. A therapeutic article of clothing for creating a magnetic field in an around a penis, the clothing comprising;
a. an undergarment having an interior, an exterior side, a front side and a back side;
b. a therapeutic magnetic pouch comprising a closed end, the closed end further comprising an inner and an outer surface, an open end, a side wall, the side wall joining the closed end and the open end together to create a pocket of generally tubular shape defined by the side wall and the closed end, at least one magnet placed on the inner surface of the closed end with the magnetic field directed toward the open end of the pouch and a complementary fastening element attached to the side wall; and
c. a complementary fastening along the length of the center of the front and fastening velcro as a waistband inside in the front.

22. The article of clothing of claim 21 where the strength of the magnetic field produced by the at least one magnet is effective in increasing blood circulation in the penis and causing an erection.

23. The article of clothing of claim 22 where the at least one magnet has a steel backing opposite the direction of the magnetic field.

24. The article of clothing of claim 21 where the pouch further comprises a steel foil attached to an outer side of the pouch opposite the complementary fastening element.

25. The article of clothing of claim 21 where the side wall is comprised of magnetic material.

26. The article of clothing of claim 25 where the strength of the magnetic field produced by the at least one magnet and the side wall is effective in increasing blood circulation in the penis and causing an erection.

27. The article of clothing of claim 26 where the at least one magnet produces a stronger magnetic field that the magnetic field produced by the side wall.

28. The article of clothing of claim 21 wherein only discrete portions of the sidewall comprise a magnetic material.

29. The article of clothing of claim 28 where the discrete portions of the sidewall comprising magnetic material correspond to the course of at least one major artery or vein in the penis.

30. The article of clothing claim 29 where the strength of the magnetic field produced by the at least one magnet and the side wall is effective in increasing blood circulation in the penis and causing an erection.

31. The article of clothing of claim 30 where the at least one magnet produces a stronger magnetic field than the magnetic field produced by the side wall.

32. A method for treatment of erectile dysfunction comprising
a. exposing the penis to a static magnetic field produced by at least 2 magnets, at least one of the at least 2 magnets being positioned horizontally to the longitudinal axis of the penis and at least one of the at least 2 magnets being positioned perpendicularly to the longitudinal axis of the penis, the strength of the magnetic field generated by the at least two magnets is effective in increasing blood circulation in the penis and causing an erection;
b. maintaining the exposure until penile erection is achieved; and
c. removing the penis from the static magnetic field.

33. The method of claim 32 where the magnetic field produced by the at least one magnet positioned perpendicularly to the longitudinal axis of the penis is stronger than the magnetic field produced by the at least one magnet positioned horizontally to the longitudinal axis of the penis.
